(19) United States
(12) Patent Application Publication
(10) Pub. No.: US 2026/0355524 A1
(43) Pub. Date: Nov. 12, 2026

(54) IMPLANTABLE MEDICAL STIMULATION DEVICE FOR PERFORMING A CARDIAC PACING IN A PATIENT

(71) Applicant: BIOTRONIK SE & Co. KG, Berlin (DE)

(72) Inventors: Frank BECKER, Berlin (DE); Jochen PROFF, Kalchreuth (DE)

(73) Assignee: BIOTRONIK SE & Co. KG, Berlin (DE)

(21) Appl. No.: 19/127,942

(22) PCT Filed: Nov. 6, 2023
(86) PCT No.: PCT/EP2023/080782

§ 371 Date: May 7, 2025

(30) Foreign Application Priority Data
Nov. 16, 2022 (EP) 22207752.1

(51) Int. Cl. A61N 1/365

(52) U.S. Cl. CPC A61N 1/36585; A61N 1/36521; A61N 1/36542

(57) ABSTRACT

Implantable medical stimulation device for performing cardiac pacing includes a generator device including processing circuitry for processing cardiac sense signals and generating cardiac stimulation signals, and an electrode arrangement for sensing cardiac signals and outputting cardiac stimulation signals. The processing circuitry configured to: (1) perform a rate-adaptive cardiac stimulation in which a paced heart rate is adjusted based on a load state of the patient; (2) obtain a momentary impedance curve indicative of a measured impedance during a cardiac cycle and compare said momentary impedance curve to a stored rest reference curve; and (3) compare said momentary impedance curve to a stored load reference curve indicative of a reference impedance curve in a load state of the patient and, based on the comparison of said momentary impedance curve to the load reference curve, to adjust said adaption of the paced heart rate and/or derive and store a statistical parameter value.

### CROSS-REFERENCE TO RELATED APPLICATIONS

This application is the United States National Phase under 35 U.S.C. § 371 of PCT International Patent Application No. PCT/EP2023/080782, filed on Nov. 6, 2023, which claims the benefit of European Patent Application No. 22207752.1, filed on Nov. 16, 2022, the disclosures of which are hereby incorporated by reference herein in their entireties.

### TECHNICAL FIELD

The instant invention relates to an implantable medical stimulation device for performing a cardiac pacing in a patient according to the preamble of claim 1 and to a method for operating an implantable medical stimulation device for performing a cardiac pacing in a patient.

### BACKGROUND

An implantable medical stimulation device of this kind comprises a generator device comprising processing circuitry for processing cardiac sense signals and generating cardiac stimulation signals. In addition, the implantable medical stimulation device comprises an electrode arrangement for sensing cardiac signals and outputting cardiac stimulation signals.

An implantable medical stimulation device of the type concerned herein may for example be configured for subcutaneous implantation, such that the generator device during implantation is subcutaneously implanted in a patient. Herein, an electrode arrangement may be provided on one or multiple electrode leads extending from the generator device and a region, for example, into the patient's heart.

During operation of the implantable medical stimulation device, the processing circuitry of the generator device generates and outputs, using the electrode arrangement, cardiac stimulation signals in order to provide for a cardiac pacing. Herein, the processing circuitry is configured to perform a rate adaptive cardiac stimulation in which a paced heart rate is adjusted based on a load state of the patient. If it for example is found that the patient is physically active such that an increased heart rate is indicated, a paced heart rate may be adapted such that a pacing at an increased heart rate occurs.

In particular, for the rate adaptive cardiac stimulation the processing circuitry is configured to obtain, based on electrical excitation signals output using the electrode arrangement and electrical response signals received in response to the electrical excitation signals, a momentary impedance curve indicative of a measured impedance during a cardiac cycle, to compare the momentary impedance curve to a stored rest reference curve indicative of a reference impedance curve in a rest state of the patient and to perform an adaption of the paced heart rate based on the comparison of the momentary impedance curve to the stored rest reference curve.

A rate adaptive cardiac stimulation scheme is for example described in U.S. Pat. No. 6,263,243. Within the rate adaptive cardiac stimulation scheme a momentary impedance curve is measured and compared to a reference curve. Based on a deviation of the momentary impedance curve with respect to the reference curve the paced heart rate is adapted such that a pacing at an increased heart rate occurs, wherein the heart rate increase depends on the deviation of the momentary impedance curve from the reference curve. If for example only a slight deviation is detected, the heart rate is only slightly adapted. If a large deviation is detected, in contrast, a large heart rate adaption takes place.

In a current rate adaptive cardiac stimulation scheme, as for example described in U.S. Pat. No. 6,263,243, a maximum heart rate may be programmed, beyond which the paced heart rate may not increase. If a large deviation of the momentary impedance curve with respect to the stored reference curve is identified, potentially the paced heart rate may be increased up to the programmed maximum heart rate, but not above the programmed maximum heart rate.

For setting the maximum heart rate, for example the so-called Astrand formula may be employed, in which the allowable maximum heart rate is set according to the value 220 minus the age of the patient. However, for patients with certain heart conditions, such as a chronic cardiac insufficiency, it may occur that an increase of the heart rate in a load state of the patient does not benefit the physical condition of the patient, but rather has an adverse effect in that the cardiac function deteriorates with increasing heart rate. This is due to the finding that cardiac contractility, for patients with chronic cardiac insufficiency, may not increase with increasing heart rate (as it normally would occur), but may deteriorate beyond an upper bound of the heart rate.

The present disclosure is directed toward overcoming one or more of the above-mentioned problems, though not necessarily limited to embodiments that do.

### SUMMARY

It is an object of the instant invention to provide an implantable medical stimulation device for performing a cardiac pacing in a patient and a method for operating an implantable medical stimulation device which in a reliable manner allow for controlling operation in particular for a rate adaptive cardiac stimulation and, potentially, for deriving diagnostic information.

This object is achieved by means of an implantable medical stimulation device comprising the features of claim 1.

Accordingly, the processing circuitry is configured to compare the momentary impedance curve to a stored load reference curve indicative of a reference impedance curve in a load state of the patient and, based on the comparison of the momentary impedance curve to the load reference curve, to at least one of the adjust the adaption of the paced heart rate and derive and store a statistical parameter value.

The implantable medical stimulation device is configured to provide for a cardiac pacing, wherein for performing the cardiac pacing the processing circuitry is configured to carry out a rate adaptive cardiac stimulation scheme. Within the rate adaptive cardiac stimulation, a paced heart rate is adjusted based on a load state of the patient, such that with changing load of the patient the paced heart rate is adapted in order to adjust the heart rate to the actual load condition of the patient.

If for example a patient is physically active and hence is in a load state of increased physical load, the paced heart rate is adapted such that the heart rate is increased, hence adapting to the current load state of the patient. Likewise, if a patient is mentally active and hence is in a mental load state, the paced heart rate may be adapted accordingly and likewise may be increased. By performing a rate adaptive cardiac stimulation, hence, a paced rate is not fixed, but is adaptively changed during operation of the implantable medical stimulation device.

For performing the rate adaptive cardiac stimulation, a momentary impedance curve is compared to a stored rest reference curve. Based on a deviation of the momentary impedance curve to the stored rest reference curve the paced heart rate is set, wherein a large deviation of the momentary impedance curve to the stored reference curve causes a large adaption of the heart rate, whereas a slight deviation of the momentary impedance curve to the stored rest reference curve causes only a slight modification of the heart rate.

The processing circuitry hence stores a rest reference curve which is indicative of a reference impedance curve in a rest state of the patient. The rest reference curve may be determined and stored during a calibration phase and may be repeatedly adapted during operation of the implantable medical stimulation device. The stored rest reference curve hence provides for a reference of an impedance curve during a cardiac cycle in a rest state of the patient, such that based on a comparison of a momentary impedance curve to the stored reference curve a deviation from the rest state may be identified.

Herein, the processing circuitry is configured to store, in addition to the rest reference curve, a load reference curve which is indicative of a reference impedance curve in a load state of the patient. For example, the processing circuitry may store multiple load reference curves, which may relate to different load states of the patient, for example a physical load state and a mental state. Hence, in the processing circuitry different reference curves, relating to the rest state and at least one load state of the patient, are maintained.

Based on the load reference curve, operation of the implantable medical stimulation device may be adapted, and/or diagnostic information may be derived.

In particular, a momentary impedance curve is compared, in addition to the comparison to the rest reference curve, to the stored load reference curve. Based on the comparison the adaption of the stimulated heart rate may be adapted, and/or statistical information may be derived in order to derive potentially relevant diagnostic information.

By comparing the momentary impedance curve to the stored load reference curve, it in particular may become possible to prevent an increase of the paced heart rate up to a programmed maximum heart rate in case it is found that a further increase of the heart rate from a current value does not benefit the patient, but rather may have an adverse effect on the patient. In particular, for a patient having chronic cardiac insufficiency, it may be the case that the paced heart rate should not be increased up to the programmed maximum heart rate, but rather a lower bound for the heart rate may exist beyond which the heart rate should not increase. This bound may be identified based on the comparison of the momentary impedance curve to the stored load reference curve.

Alternatively or in addition, based on the comparison of the momentary impedance curve to the load reference curve one or multiple statistical parameters, such as parameters relating to the deviation of the momentary impedance curve from the stored load reference curve, may be derived and stored and may for example be communicated to an external device, for example within the context of a home monitoring system, such that based on the derived statistical parameter values diagnostic information may be obtained.

In one embodiment, the processing circuitry is configured to derive a first difference parameter value indicative of a difference between the momentary impedance curve and the stored rest reference curve and to perform the adaption of the paced heart rate based on the first difference parameter value. The first difference parameter value may for example be indicative of an area in between the momentary impedance curve and the stored rest reference curve. In another embodiment, the first difference parameter value may relate to a difference between a maximum of the momentary impedance curve and the stored rest reference curve, a difference between an average or a median of the momentary impedance curve and the stored rest reference curve, or to another numerical value indicative of a deviation of the momentary impedance curve from the stored rest reference curve.

In one embodiment, the processing circuitry is configured, based on the first difference parameter value, to increase the paced heart rate in comparison to a heart rate at rest. Based on the first difference parameter value, the heart rate adaption may be set, a large value for the first difference parameter value indicating a large deviation of the momentary impedance curve to the stored rest reference curve and hence causing a large adaption of the paced heart rate, and a small value for the first difference parameter value indicating a small deviation of the momentary impedance curve to the stored rest reference curve and hence causing a small modification of the paced heart rate. In one embodiment, the processing circuitry is configured to derive a second difference parameter value indicative of a difference between the momentary impedance curve and the stored load reference curve. Similar to the first difference parameter value, the second difference parameter value may for example be indicative of an area in between the momentary impedance curve and the stored load reference curve, or may relate to a difference between a maximum of the momentary impedance curve and the stored load reference curve, a difference between an average or a median of the momentary impedance curve and the stored load reference curve, or to another numerical value indicative of a deviation of the momentary impedance curve from the load rest reference curve.

In one embodiment, the processing circuitry is configured to adjust the adaption of the paced heart rate based on the second difference parameter value. In particular, the processing circuitry may be configured, based on the second difference parameter value, to inhibit a further increase of the paced heart rate above a current value of the paced heart rate or to cause a decrease of the paced heart rate below the current value of the paced heart rate. For example, the processing circuitry may be configured to inhibit a further increase of the paced heart rate or cause a decrease of the paced heart rate based on a comparison of the second difference parameter value to a predefined threshold value.

Within the heart rate adaption scheme, based on a load state of the patient the heart rate is increased in comparison to a heart rate at rest. If the patient is very active (mentally or physically) and hence a large deviation of the momentary impedance curve with respect to the stored rest reference curve is detected, hence indicative of a state of large load, the regular algorithm for the heart rate adaption may cause an increase of the heart rate up to the programmed maximum heart rate. However, the comparison of the momentary impedance curve to the stored load reference curve and hence the derivation of the second difference parameter value allows for a control for preventing an excessive increase of the paced heart rate, in particular for patients having a cardiac state, for example a chronic cardiac insufficiency, for which the heart rate should not be excessively increased and for which the programmed maximum heart rate may lie beyond a medically advisable bound for the paced heart rate.

It has been found that, for patients having an adverse cardiac state such as a chronic cardiac insufficiency, in a load state the impedance curve may first approach the stored load reference curve with increasing heart rate. If, however, a certain bound is reached the momentary impedance curve may start to strongly deviate from the stored load reference curve, which may be detected based on the second difference parameter value which is indicative of the deviation of the momentary impedance curve from the stored load reference curve. If, in a load state, the momentary impedance curve is close to the stored load reference curve and hence a deviation of the momentary impedance curve to the stored load reference curve is small, it may be assumed that the patient is in good condition and the paced heart rate may further be increased. If however during the adaption of the heart rate it is found that the momentary impedance curve largely deviates from the stored load reference curve, this may be indicative of a situation in which in a further increase of the heart rate has an adverse effect on the patient, causing a deterioration of the contractility of the heart, such that a further increase of the paced heart rate should be inhibited or even reversed in order to bring back the patient into a state in which the heart may regularly function.

For example, the second difference parameter value, for example relating to an area in between the momentary impedance curve and the stored load reference curve, may be compared to a threshold. If the second difference parameter value becomes larger than the threshold, a further increase of the paced heart rate may be inhibited, or the paced heart rate may even be decreased. The threshold may be preprogrammed or may be adaptively set during operation, for example based on an adaption of the stored rest reference curve or the stored load reference curve during operation.

In one embodiment, the processing circuitry is configured to store, as the statistical parameter value, at least one of the second difference parameter value, a parameter value derived based on the second difference parameter value, and a maximum admissible heart rate value determined based on the second difference parameter value. For example, during the heart rate adaption the bound at which a further increase of the heart rate is inhibited may be stored and output as a statistical parameter value. The bound stored and output in this way is indicative of a state of the patient, in particular a cardiac state of the patient, for example in relation to a chronic cardiac insufficiency. Based on the statistical parameter value, hence diagnostic conclusions may be drawn, for example by monitoring a change over time of the statistical parameter value. For example, a decrease of the bound beyond which the paced heart rate during the automatic heart rate adaption should not be increased (at which a further increase is inhibited) over time may indicate a worsening of a chronic cardiac insufficiency condition of a patient.

In one embodiment, the processing circuitry is configured to store at least two load reference curves associated with at least two different defined load states. For example, the processing circuitry may be configured to store load reference curves relating to a mental load state and a physical load state, the load reference curves relating to reference impedance curves as they normally are found during the associated load state.

Herein, the processing circuitry in one embodiment is configured to identify a load state out of at least two defined load states based on the comparison of the momentary impedance curve to the stored rest reference curve. A momentary impedance curve, in a particular load state, may deviate from the stored rest reference curve in a predefined manner. Based on the comparison of the momentary impedance curve to the stored rest reference curve hence it may be concluded in which load state the patient currently is in. Based on the identified load state, for example a mental load state or a physical load state, the processing circuitry is configured to select one out of the at least two load reference curves for comparison to the momentary impedance curve, in particular to derive the second difference parameter value.

In one possible scheme, the processing circuitry may determine whether a momentary impedance curve deviates, by more than a certain margin, for example by more than a predefined area, from the rest reference curve. If this is the case, it is identified that the patient is in a load state. In addition, the processing circuitry may take an output value of at least one motion sensor into account. The motion sensor may indicate whether the patient is in a physical state of motion. If the motion sensor indicates that the patient is in a physical state of motion, the processing circuitry assumes that the identified load state is a physical load state. If the motion sensor does not indicate that the patient is in a state of physical motion, the processing circuitry instead assumes that the patient is in a mental load state. Accordingly, an associated load reference curve associated with the physical load state or the mental load state may be used for comparison to the momentary impedance curve in order to derive the second difference parameter value.

In one embodiment, the processing circuitry is configured, in a calibration phase, to derive and store at least two load reference curves indicative of a mental load state and a physical load state. During the calibration phase, hence the load reference curves are derived and stored, such that a set of different load reference curves is obtained which may be used during subsequent operation of the implantable medical stimulation device. The load reference curves may be continuously adapted during operation of the implantable medical stimulation device.

For recording the different load reference curves, upon implantation a calibration phase may be carried out, in which it is observed whether, over a substantial period of time, a stable deviation of the momentary impedance curve with respect to a stored rest reference curve is observed. If this is the case, it can be identified for example based on an output of a motion sensor whether the load state is a mental load state or a physical load state, and based on this information a load reference curve relating to a mental load state respectively a physical load state may be stored.

It herein may be determined whether the patient is in a stable load state for example by computing a running average of a difference area of the momentary impedance curve with respect to the rest reference curve. If the running average is stable, e.g. it exhibits a standard deviation smaller than a predefine bound over some time, it can be assumed that the patient is in a stable load state, such that based on an average of impedance curves as recorded during the stable load state the load reference curve may be determined and stored.

During calibration, herein, load reference curves for different load states are recorded, in particular relating to a physical load state and a mental load state.

In one embodiment, the processing circuitry is configured, in the calibration phase, to derive and store load reference curves for different load states and different stimulation event sequences. During operation of an implantable medical stimulation device for causing a cardiac pacing, different sequences of pacing events and intrinsic cardiac contraction events may occur. For example, an intrinsic atrial contraction may be followed by an intrinsic ventricular contraction, a paced atrial contraction may be followed by an intrinsic ventricular contraction, an intrinsic atrial contraction may be followed by a paced ventricular contraction, or a paced atrial contraction may be followed by a paced ventricular contraction. These are denoted as event sequences. For the different event sequences different load reference curves may be obtained, indicating in each case a particular impedance reference curve which may be dependent on the particular event sequence. Hence, during calibration a set of different load reference curves may be stored indicative of different load states and in addition associated with the different event sequences. For example, for each event sequence a pair of a mental load reference curve and a physical load reference curve may be stored.

For deriving impedance information and for monitoring an electrical impedance value, the processing circuitry, in one embodiment, is configured to generate electrical excitation signals which are output by means of the electrode arrangement. In reaction to outputting the electrical excitation signals, response signals are received, which are processed by the processing circuitry of the generator device. By correlating the electrical excitation signals and the electrical response signals received in response to the electrical excitation signals, impedance information is derived and is monitored.

An electrical excitation signal generated by the processing circuitry for outputting by the electrode arrangement may for example be a current signal produced by a defined current source, in which case the electrical response signal is a voltage signal. From the excitation signal and from the associated response signal, hence, an electrical impedance may be computed, wherein the impedance calculation is repeated throughout a cardiac cycle or a predefined portion of a cardiac cycle, in particular a partial relating to a ventricular contraction event.

In another embodiment, an electrical excitation signal generated by the processing circuitry for outputting by the electrode arrangement is a voltage signal produced by a defined voltage source, in which case the electrical response signal is a current signal. Again, from the electrical excitation signal and from the associated electrical response signal an impedance value may be computed, wherein the impedance calculation is repeated throughout a cardiac cycle or a predefined portion of a cardiac cycle, in particular a partial relating to a ventricular contraction event.

In one embodiment, electrical excitation signals as generated by the processing circuitry to be output by the electrode arrangement are biphasic electrical pulse signals. Such biphasic electrical pulse signals may be formed by a first pulse section having a positive amplitude and a consecutive, second pulse section having a negative amplitude, or vice versa.

In another aspect, a method for operating an implantable medical stimulation device for performing a cardiac pacing in a patient, the method comprising: providing a generator device comprising processing circuitry for processing cardiac sense signals and generating cardiac stimulation signals; providing an electrode arrangement for sensing cardiac signals and outputting cardiac stimulation signals; performing, using the processing circuitry, a rate-adaptive cardiac stimulation in which a paced heart rate is adjusted based on a load state of the patient, wherein the processing circuitry obtains a momentary impedance curve indicative of a measured impedance during a cardiac cycle, compares said momentary impedance curve to a stored rest reference curve indicative of a reference impedance curve in a rest state of the patient and performs an adaption of the paced heart rate based on the comparison of the momentary impedance curve to the stored rest reference curve; and comparing, using the processing circuitry, said momentary impedance curve to a stored load reference curve indicative of a reference impedance curve in a load state of the patient; and at least one of adjusting said adaption of the paced heart rate and deriving and storing a statistical parameter value based on the comparison of said momentary impedance curve to the load reference curve.

The advantages and advantageous embodiments described above for the implantable medical stimulation device equally apply also to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The idea of the invention shall subsequently be described in more detail with reference to the embodiments shown in the figures. Herein:

FIG. 1 shows a schematic view of an implantable medical stimulation device having a generator device and electrode leads;

FIG. 2A shows a schematic drawing of the implantable medical stimulation device of FIG. 1, illustrating an impedance measurement during operation;

FIG. 2B shows an impedance curve as measured during a cardiac cycle or a predefined portion of a cardiac cycle, in particular relating to a ventricular contraction event;

FIG. 3A shows a rest reference curve indicative of a reference impedance curve in a rest state of the patient;

FIG. 3B shows an example of a momentary impedance curve in a mental load state of the patient;

FIG. 3C shows an example of the difference between a momentary impedance curve in a mental load state of the patient and a momentary impedance curve in a physical load state of the patient;

FIG. 4A shows the rest reference curve of FIG. 3A;

FIG. 4B shows the impedance curve in a mental load state of FIG. 3B, and in addition a momentary impedance curve relating to an adverse state of the patient;

FIG. 4C shows the impedance curve in a physical load state of FIG. 3C, and in addition a momentary impedance curve relating to an adverse state of the patient;

FIG. 5A shows a load reference curve relating to a mental load state and a momentary impedance curve of an expected stable mental load state;

FIG. 5B shows the load reference curve of FIG. 5A, together with a momentary impedance curve in an adverse state of the patient; (mental load)

FIG. 6A shows a load reference curve relating to a physical load state and a momentary impedance curve of an expected stable physical load state;

FIG. 6B shows the load reference curve of FIG. 6A, together with a momentary impedance curve in an adverse state of the patient (physical load); and

FIG. 7 shows an example of a rest reference curve, a load reference curve and a momentary impedance curve relating to an adverse state of the patient.

### DETAILED DESCRIPTION

Subsequently, embodiments of the invention shall be described in detail with reference to the drawings. In the drawings, like reference numerals designate like structural elements.

It is to be noted that the embodiments are not limiting for the invention, but merely represent illustrative examples.

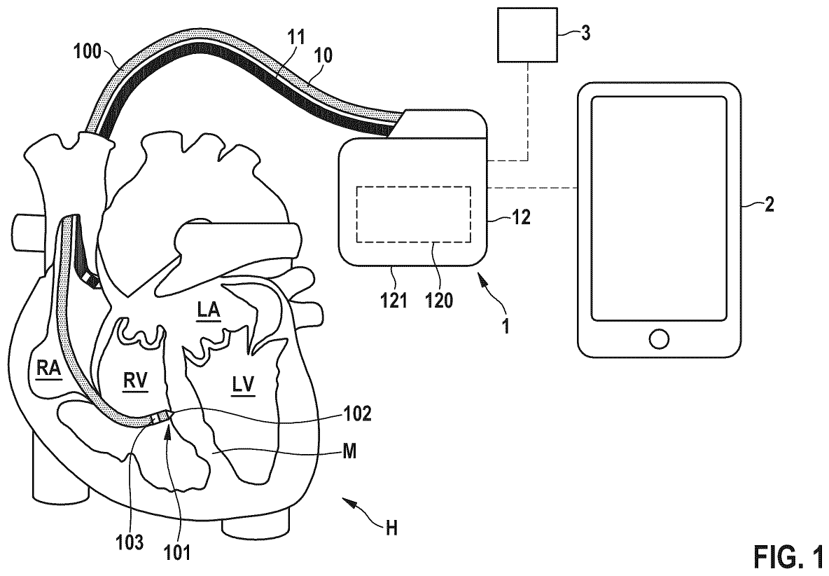

FIG. 1 shows, in a schematic drawing, the human heart H comprising the right atrium RA, the right ventricle RV, the left atrium LA and the left ventricle LV. An implantable medical stimulation device 1 is implanted in a patient, the implantable medical stimulation device 1 comprising a generator 12 connected to leads 10, 11 extending from the generator 12 through the superior vena into the patient's heart H. By means of the leads 10, 11, electrical signals for providing a pacing action in the heart H shall be injected into intra-cardiac tissue M potentially at different locations within the heart, and sense signals may be received.

An implantable medical stimulation device 1 as concerned herein may generally be a cardiac stimulation device such as a cardiac pacemaker device. A stimulation device of this kind may comprise a generator 12, as shown in FIG. 1, which may be subcutaneously implanted in a patient at a location remote from the heart H, one or multiple leads 10, 11 extending from the generator 12 into the heart H for emitting stimulation signals in the heart H or for obtaining sense signals at one or multiple locations from the heart H. The leads 10, 11 each form a generally longitudinal, tubular body 100, which reaches into the heart H and is anchored at a location of interest within the heart H.

In the embodiment of FIG. 1, the electrode leads 10, 11 may comprise electrode poles 102, 103 at or in the vicinity of the distal end 101 of the respective lead body 100, the electrode poles 102, 103 together forming an electrode pole arrangement for sensing cardiac signals and for outputting cardiac stimulation signals in the course of operation of the implantable medical stimulation device 1 for performing a cardiac pacing action. Another electrode pole herein may be formed by the housing 121 of the generator device 12, the housing 121 providing a counter-electrode for any one of the electrode poles 102, 103 of the electrode leads 10, 11.

As schematically shown in FIG. 1, the implantable medical stimulation device 1 may be in communication with an external device 2 which is external to the patient and for example is part of a home monitoring system or represents a programming device for programming the implantable medical stimulation device 1.

In addition, a motion sensor 3 may be integrated into the generator device 12 or may be implemented at another location in or on the patient, the motion sensor 3 being in communicative connection with the generator device 12 such that a processing circuitry 120 of the generator device 12 receives signals of the motion sensor 3 and evaluates signals in order to derive information with respect to a motion state of the patient.

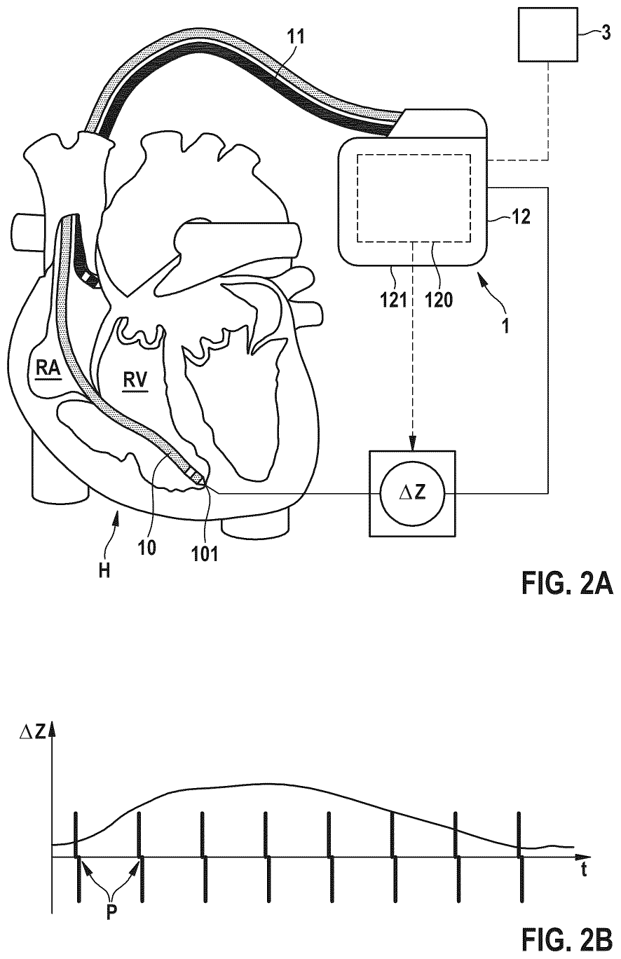

Referring now to FIG. 2A, the implantable medical stimulation device 1 shall be configured to perform a pacing action in which cardiac activity is paced, in particular in scenarios in which intrinsic cardiac activity does not reliably occur. During operation, the processing circuitry 120 of the generator device 12 may for example be configured to sense and process cardiac sense signals, received via the electrode pole arrangement, and generate cardiac stimulation signals to be output via the electrode pole arrangement in response to sensed and processed cardiac signals.

The generator device 12 herein shall implement a cardiac rate adaption scheme, also denoted as Closed-Loop Stimulation (in short CLS), in which a paced heart rate is adapted based on a load state of the patient. This is based on the general principle that the paced heart rate should increase if the patient is physically and/or mentally active and hence is in a load state.

For performing the rate adaption, the processing circuitry 120 of the generator device 12 is configured to record an impedance curve, as it is shown in an example in FIG. 2B. For this, the processing circuitry 120 repeatedly measures impedance values during a cardiac cycle or a predefined portion of the cardiac cycle by generating and outputting electrical excitation signals and receiving, in response, electrical response signals. By correlating the electrical excitation signals to the electrical response signals, an impedance value is derived relating to an intracardiac impedance e.g. in a certain portion of a cardiac cycle, in particular relating to ventricular contractions.

As shown in FIG. 2B, the processing circuitry 120 may for example produce, as electrical excitation signals, biphasic pulses P to be output by a pair of electrode poles 102, 103, 121, for example the tip electrode pole 102 and the pole formed by the housing 121.

In one embodiment, the electrical excitation signals as generated by the processing circuitry 120 to be output by a pair of electrode poles 102, 103, 121 are current signals which are generated by a controlled current source. In this case, the electrical response signals are voltage signals. In another embodiment, the electrical excitation signals as generated by the processing circuitry 120 to be output by a pair of electrode poles 102, 103, 121 are voltage signals which are generated by a controlled voltage source. In this case, the electrical response signals are current signals.

It has been found that the intracardiac impedance, in a particular time window in relation to a QRS complex, exhibits an especially significant dependency on the patient's load state. Hence, by measuring the impedance it may be derived whether a patient's state differs from a rest state, such that based on a deviation of a momentary impedance curve as measured by the processing circuitry 120 a paced heart rate may be adjusted.

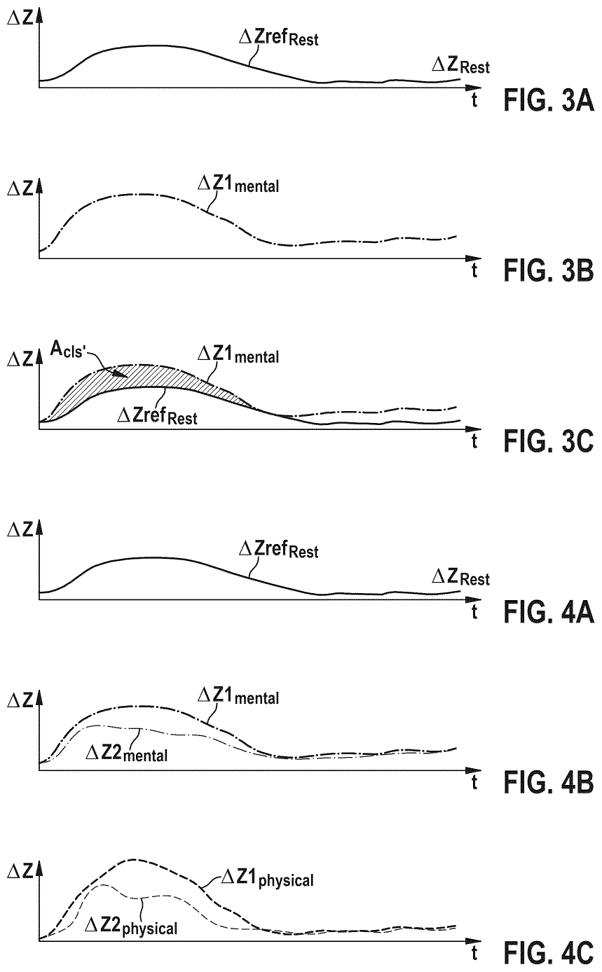

Referring now to FIGS. 3A to 3C, at rest an impedance curve may exhibit a particular shape, for example a somewhat flattened shape, yielding a rest reference curve ΔZref_{Rest} as shown in FIG. 3A. In contrast, in a load state, for example a state relating to increased mental activity, a momentary impedance curve ΔZ1ₘₑₙₜₐₗ may substantially differ, as shown in FIG. 3B for the momentary impedance curve ΔZ1ₘₑₙₜₐₗ by itself and in FIG. 3C according to the momentary impedance curve ΔZ1ₘₑₙₜₐₗ together with the rest reference curve ΔZref_{Rest}.

A deviation of the momentary impedance curve ΔZ1ₘₑₙₜₐₗ from the rest reference curve ΔZref_{Rest} herein is quantified by a difference parameter value for example relating to the area A_{cls} in between the momentary impedance curve ΔZ1ₘₑₙₜₐₗ and the rest reference curve ΔZref_{Rest}.

For performing a rate adaption, a rest reference curve ΔZref_{Rest} as shown in FIG. 3A may for example be recorded in an initial calibration phase and may potentially be adapted according to changing conditions during operation of the implantable medical stimulation device 1. During operation of the implantable medical stimulation device 1 for performing a pacing action, then, a momentary impedance curve ΔZ1ₘₑₙₜₐₗ, as shown in an example in FIG. 3B, is compared to the rest reference curve ΔZref_{Rest}, and a difference parameter value A_{cls} is derived indicating a deviation of the momentary impedance curve ΔZ1ₘₑₙₜₐₗ from the rest reference curve ΔZref_{Rest}. Based on the difference parameter value A_{cls}, then, a rate adaption is performed, wherein the rate adaption depends on the amount of the deviation and hence the amount of the difference parameter value.

Generally, if a large deviation of the momentary impedance curve ΔZ1ₘₑₙₜₐₗ with respect to the rest reference curve ΔZref_{Rest} is identified, the paced heart rate is successively adapted to increase the paced heart rate. If only a small deviation of the momentary impedance curve ΔZ1ₘₑₙₜₐₗ with respect to the rest reference curve ΔZref_{Rest} is identified, in contrast the paced heart rate is only slightly adapted.

Referring now to FIGS. 4A to 4C, generally an increase in the heart rate causes an increase in the contractility of the heart. However, if the heart suffers from chronic cardiac insufficiency, at a particular bound a further increase in the heart rate may not cause a further increase in the contractility, but rather may have an adverse effect in that contractility deteriorates.

It has been found that this also can be observed in the momentary impedance curve. FIG. 4A again shows the rest reference curve ΔZref_{Rest}. FIG. 4B shows a momentary impedance curve ΔZ1ₘₑₙₜₐₗ in a mental load state for a heart not suffering from chronic cardiac insufficiency and a momentary impedance curve ΔZ2ₘₑₙₜₐₗ for a heart suffering from chronic cardiac insufficiency. FIG. 4C shows a momentary impedance curve ΔZ1_{physical} in a physical load state for a heart not suffering from chronic cardiac insufficiency and a momentary impedance curve ΔZ2_{physical} for a heart suffering from chronic cardiac insufficiency. As can be seen, for a patient suffering from chronic cardiac insufficiency a momentary impedance curve ΔZ2ₘₑₙₜₐₗ, ΔZ2_{physical} at an increased heart rate is observed which substantially differs from the impedance curve ΔZ1ₘₑₙₜₐₗ, ΔZ1_{physical} normally found in a patient not suffering from chronic cardiac insufficiency.

This has the effect that, when during a regular adaption of the paced heart rate according to a detected load state the heart rate is successively increased, an improvement in the contractility of the heart may not be observed in all patients, but rather for patients suffering from an adverse heart condition, in particular a chronic cardiac insufficiency, the contractility may not further improve beyond a certain bound, but in contrast may deteriorate. Hence, an increase of the heart rate beyond a certain bound during an automatic rate adaption may potentially not be beneficial but may rather have an adverse effect to the patient.

Hence, it may be desirous to avoid an automatic increase of the paced heart rate up to a programmed maximum heart rate in particular for patients suffering from an adverse heart condition, such as chronic cardiac insufficiency.

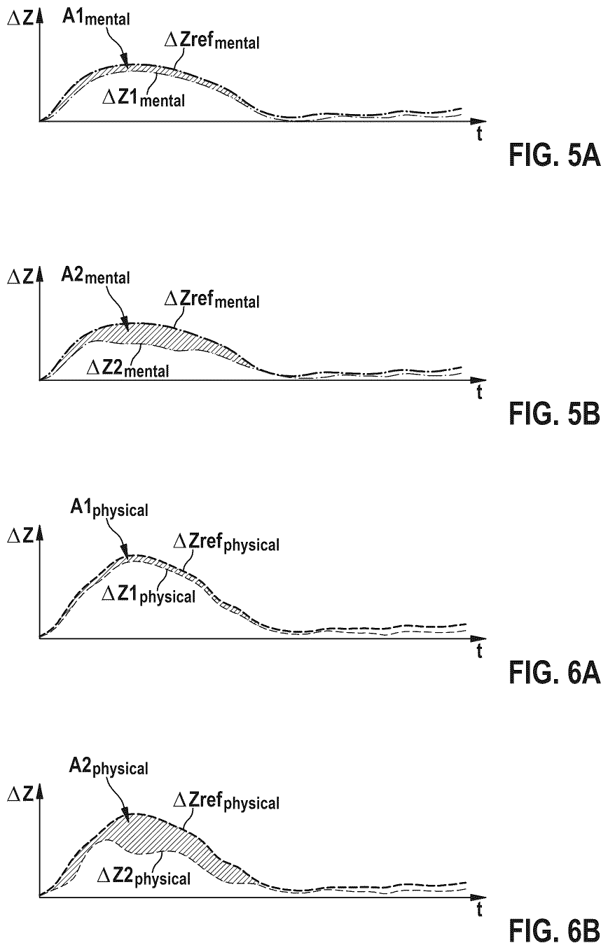

It herein is proposed to store not only a rest reference curve, but in addition one or multiple load reference curves, for example a load reference curve ΔZrefₘₑₙₜₐₗ relating to a mental load state and a load reference curve ΔZref_{physical} relating to a physical load state, as it is shown in FIGS. 5A, 5B for the mental load state and FIGS. 6A, 6B for the physical load state.

Herein, as impedance curves may differ based on a specific event sequence, in one embodiment different load reference curves may be stored for different event sequences. For example, a first event sequence for a cardiac cycle may be an intrinsic atrial contraction followed by an intrinsic ventricular contraction (As-Vs), another event sequence may be a paced atrial contraction followed by an intrinsic ventricular contraction (Ap-Vs), another event sequence may be an intrinsic atrial contraction followed by a paced ventricular contraction (As-Vp), and yet another event sequence may be a paced atrial contraction followed by a paced ventricular contraction (Ap-Vp). For each event sequence a pair of load reference curves relating to a mental load state and a physical load state may be stored.

In addition, it is conceivable to store multiple load reference curves for example for different physical load states, for example a state of medium physical activity and a state of excessive physical activity.

The different load reference occurs ΔZrefₘₑₙₜₐₗ, ΔZref_{physical} may be stored for example in an initial calibration phase and may be updated regularly during operation. For example, during an initial calibration phase it may be determined whether the patient is in a stable load state, in which for example a deviation from a stored rest reference curve, as illustrated in FIG. 3C, is stable over a substantial portion of time. If such a stable load state is identified, based on information provided by the motion sensor 3 it may be identified whether the load state relates to a mental load state (indicated by an output of the motion sensor indicating no substantial physical movement of the patient) or in a physical load state (indicated by an output of the motion sensor 3 indicating a substantial physical motion of the patient). By recording and averaging impedance curves over a multiplicity of cardiac cycles, then, a respective load reference curve may be determined and stored.

A stability criterion for a stable load state may for example be determined according to a running average of a difference area of the rest reference curve and the momentary impedance curve as measured by the processing circuitry 120. If the running average is found to be stable, for example by exhibiting a standard deviation smaller than a predefined threshold, a stable load state is assumed.

A calibration phase may be concluded once all required reference curves are recorded and stored. Referring now to FIGS. 5A, 5B and FIGS. 6A, 6B, for a patient not suffering from an adverse heart condition such as a chronic cardiac insufficiency, the momentary impedance curve ΔZ1ₘₑₙₜₐₗ, ΔZ1_{physical} may approach towards the load reference curve ΔZrefₘₑₙₜₐₗ, ΔZref_{physical}, as this is illustrated in FIG. 5A for the mental load state and FIG. 6A for the physical load state, if during the heart rate adaption the heart rate is automatically adapted to match the load state.

If however the patient suffers from an adverse heart condition such as a chronic cardiac insufficiency, the momentary impedance curve ΔZ2ₘₑₙₜₐₗ, ΔZ2_{physical} may initially be found to approach towards the load reference curve ΔZrefₘₑₙₜₐₗ, ΔZref_{physical}, but at a certain heart rate may found to substantially deviate in shape from the load reference curve ΔZrefₘₑₙₜₐₗ, ΔZref_{physical}, as this can be seen in FIG. 5B for the mental load state and FIG. 6B for the physical load state.

This occurrence of a deviation may be evaluated in order to adjust the automatic heart rate adaption. In particular, a second difference parameter value corresponding to the area A1ₘₑₙₜₐₗ, A2ₘₑₙₜₐₗ, A1_{physical}, A2_{physical} in between the momentary impedance curve ΔZ1ₘₑₙₜₐₗ, ΔZ1_{physical}, ΔZ2ₘₑₙₜₐₗ, ΔZ2_{physical} and the respective load reference curve ΔZrefₘₑₙₜₐₗ, ΔZref_{physical} may be determined, the second difference parameter value being indicative of the deviation of the momentary impedance curve ΔZ1ₘₑₙₜₐₗ, ΔZ1_{physical}, ΔZ2ₘₑₙₜₐₗ, ΔZ2_{physical} from the load reference curve ΔZrefₘₑₙₜₐₗ, ΔZref_{physical} associated with the particular load state. If the second difference parameter value indicates a deviation of the momentary impedance curve ΔZ1ₘₑₙₜₐₗ, ΔZ1_{physical}, ΔZ2ₘₑₙₜₐₗ, ΔZ2_{physical} from the load reference curve ΔZrefₘₑₙₜₐₗ, ΔZref_{physical} by more than a certain margin, for example if the second difference parameter value becomes larger than a predefined threshold, it is assumed that a further improvement in the contractility of the heart cannot be achieved by further increasing the heart rate, such that a further increase of the heart rate during the heart rate adaption process is inhibited and, potentially in addition, the heart rate is decreased by a certain margin in order to improve contractility of the heart.

Hence, during the heart rate adaption, the second difference parameter value indicative of the deviation of the momentary impedance curve ΔZ1ₘₑₙₜₐₗ, ΔZ1_{physical}, ΔZ2ₘₑₙₜₐₗ, ΔZ2_{physical} to the load reference curve ΔZrefₘₑₙₜₐₗ, ΔZref_{physical} associated with a particular load state is monitored. If the second difference parameter value becomes large and hence a substantial deviation of the momentary impedance reference curve ΔZ1ₘₑₙₜₐₗ, ΔZ1_{physical}, ΔZ2ₘₑₙₜₐₗ, ΔZ2_{physical} from the associated load reference curve ΔZrefₘₑₙₜₐₗ, ΔZref_{physical} is identified, it is assumed that the heart rate, during the automatic heart rate adaption, should not be further increased, but rather should be kept at the current value or should be increased.

In this way, for example a bound beyond which the paced heart rate shall not be increased, is determined. The bound may be stored and for example may also be communicated to the external device 2.

By employing one or multiple load reference curves ΔZrefₘₑₙₜₐₗ, ΔZref_{physical}, hence, the process of the automatic heart rate adaption scheme may be adjusted by monitoring up to which heart rate an improvement in the contractility of the heart can be achieved and by inhibiting a further increase if it is found that no further improvement in the contractility can be achieved.

Alternatively or in addition, based on the comparison of the momentary impedance curve ΔZ1ₘₑₙₜₐₗ, ΔZ1_{physical}, ΔZ2ₘₑₙₜₐₗ, ΔZ2_{physical} with a stored load reference curve ΔZrefₘₑₙₜₐₗ, ΔZref_{physical}, statistical parameter values may be recorded, for example relating to a bound beyond which a further improvement of the contractility cannot be achieved. From these statistical parameter values, which may be communicated to an external device 2 for example within a home monitoring system, diagnostic conclusions may be drawn, in that the statistical parameter values may be stored over time and hence an evolution of the statistical parameter values over time may be observed.

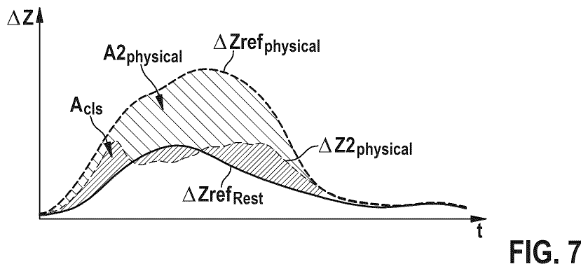

Referring now to FIG. 7, when a momentary impedance curve ΔZ2_{physical} substantially deviates from a load reference curve ΔZref_{physical} associated with a current load state, this may also be due to a reduced load on the patient, the patient hence approaching the rest state. In order to distinguish a deviation from the load reference curve ΔZref_{physical} which is due to a deterioration of the contractility of the heart due to an adverse heart condition from a deviation which is due to a reduced load, the first difference parameter value A_{cls} indicating the difference of the momentary impedance curve ΔZ2_{physical} with respect to the rest reference curve ΔZref_{Rest} as well as the second difference parameter value A2_{physical} indicating the difference of the momentary impedance curve ΔZ2_{physical} with respect to the load reference curve ΔZref_{physical} may be taken into account. If both difference parameter values A_{cls}, A2_{physical} are found to be large, for example larger than a predefined threshold, it can be assumed that the deviation of the momentary impedance curve ΔZ2_{physical} with respect to the load reference curve ΔZref_{physical} is due to a deterioration in the contractility due to an adverse heart condition, such that a further increase in the heart rate is inhibited or the heart rate even reduced.

The idea underlying the invention is not limited to the embodiment described above but may be implemented in an entirely different fashion.

In one embodiment, multiple load reference curves relating to different load states are stored and used in addition to a rest reference curve. Herein, multiple different load reference curves for example relating to different physical load states or different mental load states may be used and stored in the processing circuitry.

In principle, however, a single load reference curve may suffice.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical stimulation device
10 Lead
100 Lead body
101 Distal end
102 Electrode pole
103 Electrode pole
11 Lead
12 Generator
120 Processing circuitry
121 Housing
2 External device
3 Motion sensor
A_{cls} Difference parameter value
A1ₘₑₙₜₐₗ, A2ₘₑₙₜₐₗ Difference parameter value
A1_{physical}, A2_{physical} Difference parameter value
ΔZ1ₘₑₙₜₐₗ, ΔZ2ₘₑₙₜₐₗ Impedance curve at mental load
ΔZ1_{physical}, ΔZ2_{physical} Impedance curve at mental load
ΔZrefₘₑₙₜₐₗ Load reference curve for mental load
ΔZref_{physical} Load reference curve for physical load
ΔZref_{Rest} Rest reference curve
H Heart
LA Left atrium
LV Left ventricle
M Intra-cardiac tissue (myocardium)
P Excitation pulse
RA Right atrium
RV Right ventricle

1. An implantable medical stimulation device for performing a cardiac pacing in a patient, comprising:
a generator device comprising processing circuitry for processing cardiac sense signals and generating cardiac stimulation signals, and
an electrode arrangement for sensing cardiac signals and outputting cardiac stimulation signals,
wherein the processing circuitry is configured to perform a rate-adaptive cardiac stimulation in which a paced heart rate is adjusted based on a load state of the patient, wherein the processing circuitry is configured to obtain a momentary impedance curve indicative of a measured impedance during a cardiac cycle, to compare said momentary impedance curve to a stored rest reference curve indicative of a reference impedance curve in a rest state of the patient and to perform an adaption of the paced heart rate based on the comparison of the momentary impedance curve to the stored rest reference curve,
the processing circuitry is configured to compare said momentary impedance curve to a stored load reference curve indicative of a reference impedance curve in a load state of the patient and, based on the comparison of said momentary impedance curve to the load reference curve to at least one of adjust said adaption of the paced heart rate and derive and store a statistical parameter value.

2. The implantable medical stimulation device according to claim 1, wherein the processing circuitry is configured to derive a first difference parameter value indicative of a difference between the momentary impedance curve and the stored rest reference curve and to perform said adaption of the paced heart rate based on the first difference parameter value.

3. The implantable medical stimulation device according to claim 2, wherein the processing circuitry is configured to, based on the first difference parameter value, to increase said paced heart rate in comparison to a heart rate at rest.

4. The implantable medical stimulation device according to claim 1, wherein the processing circuitry is configured to derive a second difference parameter value indicative of a difference between the momentary impedance curve and the stored load reference curve.

5. The implantable medical stimulation device according to claim 4, wherein the processing circuitry is configured to adjust said adaption of the paced heart rate based on the second difference parameter value.

6. The implantable medical stimulation device according to claim 4, wherein the processing circuitry is configured, based on the second difference parameter value, to inhibit a further increase of said paced heart rate above a current value of the paced heart rate or to cause a decrease of said paced heart rate.

7. The implantable medical stimulation device according to claim 4, wherein the processing circuitry is configured to inhibit a further increase of said paced heart rate or cause a decrease of said paced heart rate based on a comparison of the second difference parameter value to a threshold value.

8. The implantable medical stimulation device according to claim 4, wherein the processing circuitry is configured to store, as said statistical parameter value, at least one of said second difference parameter value, a parameter value derived based on said second difference parameter value, and a maximum permissible heart rate value determined based on the second difference parameter value.

9. The implantable medical stimulation device according to claim 1, wherein the processing circuitry is configured to store at least two load reference curves associated with at least two different defined load states, to identify a load state out of the at least two defined load states based on the comparison of said momentary impedance curve to the stored rest reference curve and to select, based on the identification of the load state, a load reference curve of the at least two load reference curves for comparison to said momentary impedance curve.

10. The implantable medical stimulation device according to claim 9, wherein the processing circuitry is configured, for identifying said defined load state, to evaluate a signal of a motion sensor.

11. The implantable medical stimulation device according to claim 1, wherein the processing circuitry is configured, in a calibration phase, to derive and store at least two load reference curves indicative of a mental load state and a physical load state.

12. The implantable medical stimulation device according to claim 11, wherein the processing circuitry is configured, in the calibration phase, to derive and store the at least two load reference curves by averaging impedance curves as measured during a multiplicity of cardiac cycles.

13. The implantable medical stimulation device according to claim 11, wherein the processing circuitry is configured, in the calibration phase, to derive and store load reference curves for different load states and different event sequences.

14. The implantable medical stimulation device according to claim 1, wherein the processing circuitry is configured, for obtaining said momentary impedance curve, to generate electrical excitation signals and to provide the electrical excitation signals to the electrode arrangement for outputting into tissue and to receive, using the electrode arrangement, electrical response signals in response to the electrical excitation signals.

15. A method for operating an implantable medical stimulation device for performing a cardiac pacing in a patient, the method comprising:
providing a generator device comprising processing circuitry for processing cardiac sense signals and generating cardiac stimulation signals;
providing an electrode arrangement for sensing cardiac signals and outputting cardiac stimulation signals; and
performing, using the processing circuitry, a rate-adaptive cardiac stimulation in which a paced heart rate is adjusted based on a load state of the patient, wherein the processing circuitry obtains a momentary impedance curve indicative of a measured impedance during a cardiac cycle, compares said momentary impedance curve to a stored rest reference curve indicative of a reference impedance curve in a rest state of the patient and performs an adaption of the paced heart rate based on the comparison of the momentary impedance curve to the stored rest reference curve;
wherein comparing, using the processing circuitry, said momentary impedance curve to a stored load reference curve indicative of a reference impedance curve in a load state of the patient; and
at least one of adjusting said adaption of the paced heart rate and deriving and storing a statistical parameter value based on the comparison of said momentary impedance curve to the load reference curve.
